(19) United States
(12) Patent Application Publication
(10) Pub. No.: US 2006/0282133 A1
(43) Pub. Date: Dec. 14, 2006

(54) Method of Marking Biological Tissues for Enhanced Destruction by Applied Radiant Energy

(75) Inventor: Steven D Jensen, South Jordan, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO GROUP, INC., Sandy, UT (US)

(21) Appl. No.: 11/423,424

(22) Filed: Jun. 9, 2006

Related U.S. Application Data
(60) Provisional application No. 60/689,364, filed on Jun. 10, 2005.

(51) Int. Cl. A61N 5/06

(52) U.S. Cl. 607/88

(57) ABSTRACT

Methods for staining a selected tissue with a dye, stain or pigment that is attuned to absorb the energy from a radiant energy source are disclosed. The radiant energy source can be sufficient to destroy or combust radiated tissues. The dye or stain can enhance absorption of incoming radiant energy, which results in increased destruction of stained tissues and decreased destruction of underlying tissues. This method provides clinicians with the ability to selectively mark a tissue for destruction, while leaving wanted tissues generally intact. A clinician may dispense the stain with a pen and directly stain selected biological tissues, similar to the current practice of drawing current incision guides, followed by radiating the stained area with a laser that produces a wavelength that the stain readily absorbs. Optionally, a radiant energy opaque substance that can be applied adjacent the stained treatment area to protect against accidental or incidental exposure to wanted tissue. Also optionally, an oxidizing substance may be applied with the stain to further enhance the effect of this method.

### CROSS-REFERENCES TO RELATED APPLICATIONS

The present Application claims priority as a non-provisional perfection of prior Provisional Application 60/689,364, filed Jun. 10, 2005.

### FIELD OF THE INVENTION

The present invention relates to the field of laser treatment of biological tissues and more particularly relates to a method of staining or dying selected tissues for destruction by a general laser source rather than selecting a particular laser source dependent upon the type of tissue to be treated.

### BACKGROUND OF THE INVENTION

Clinicians are often confronted with the need of a device to cut or destroy various tissues. In recent years, lasers have become a more common device in the hands of medical, dental and veterinary practitioners. Lasers are effective tools for surgical removal of unwanted tissues or routine incisions. One of many advantages of laser surgery is the cauterizing effect of laser treated tissues, which creates a bloodless surgical environment. There are various types of lasers with multiple wavelengths and power outputs currently manufactured to best fit particular surgical needs. A practitioner will most likely choose a laser, or a number of lasers, that will cover as many routine procedures as possible. There are many reasons that any laser is selected for purchase, namely applicability to the physician's practice, cost, ease of use, size of unit, wavelength and power output.

Most lasers produce bands of coherent light of a very narrow wavelength. This narrow wavelength is a major limitation of the current generation of lasers. It requires that a specific type of laser must be chosen that emits a wavelength that is absorbed by a particular biological substrate; therefore, there are many types of lasers manufactured that individually cover a small portion of the electromagnetic spectrum. Each type of laser will then have a different clinical use or application than another type. This results in the clinician having more than one laser in order to adequately perform various biological procedures. There is particular effectiveness of this method in dental procedures, such as a gingivalectomy or root canal treatment, and surface dermatological treatments, such as a mole removal.

The use of a laser, however, does present one disadvantage. Since the laser is attuned to a narrow wavelength range, it is rare that the range will correspond to the most efficiently absorbed wavelength of subjected tissues. Two main situations cause this disadvantage. The first situation is that different layers of biological tissues that may need incised or treated in the same procedure will be attuned to different wavelengths, thus necessitating a laser that will treat all layers somewhat efficiently, but never precisely. This necessity results in excess energy being used to treat, or just get through, less efficiently absorbing tissue while more efficiently absorbing underlying tissue is bombarded with energy the exterior layer did not absorb. Secondly, different people will have different shades of tissue, in particular skin tone, when compared to others and on various parts of their own bodies (i.e. moles). One laser is not going to be attuned to all of these variations and, even if one were attuned to one particular patient's tissue, its effectiveness would change on the next patient and, possibly, at the instant a procedure was complete (e.g. a mole removal) before the laser could be shut down. In either case, the imprecise attunement of the laser to the tissue causes some degree of overpenetration. Overpenetration is the exposure, and destruction, of a column of tissue underlying the targeted tissue to unabsorbed radiant energy as it spills into deeper biological layers. Overpenetration typically causes a blistering effect as fluid released from the unwanted destruction of tissues is expressed through the wound caused by the procedure.

The present invention is a method of staining a given biological substrate for attunement to a given laser source, rather than the other way around as is practiced in the prior art. When employed with the methods disclosed herein, any efficient laser can be used on any biological substrate regardless of the wavelengths produced. The use of a stain also concentrates the laser's radiant energy in the stained tissues, lessening overpenetration by forcing an attunement of the tissues to the laser output. In addition, a substance that is opaque to a particular radiant energy can be applied around the stained treatment area to protect against incidental or accidental exposure of laterally located tissues to harmful radiant energy during treatment. Given the cost advantage of producing and purchasing a stain over a laser, the method of the present invention represents an extremely cost beneficial advancement in the art.

### SUMMARY OF THE INVENTION

The present invention is a method for destroying, cutting, pyrolizing or carbonizing tissue by first applying a dye, stain or pigment to biological tissues that is attuned to absorb incoming radiant energy, which results in the destruction of said tissues. As examples, the dye, stain or pigment could be indocyanine green, carbon black, FD&C Blue #2, nigrosin or others. The dye, stain or pigment may be applied by a pen, a brush, spraying, a fibrous pellet, a syringe tip, fiber syringe tip, or otherwise. The radiant energy source can be any source whose energy is absorbed by the dye, pigment or stain in order to build up heat, such as a diode laser, a gas laser, a solid state laser, non-coherent light, incandescent light, light emitting diode, plasma arc light, halogen bulb, electron beam, or otherwise. If desired, an opaque substance may be used to protect tissues, which are not to be cut or destroyed. Opaque substances could include titanium dioxide, zinc oxide, calcium carbonate, or otherwise.

The present invention represents a departure from the prior art in that the method of the present invention dictates the staining of a selected tissue with a dye, stain or pigment. This Application shall use the term “stain” to include all such dyes, pigments and stains and any compound or solution utilizing such dye, pigment or stain as an ingredient in it's combined whole. The use of the term “stain” is to be understood to include such “stains” that include a pigment or dye as its only ingredient. The stain is selected because it is attuned to absorb the energy from a given radiant energy source, rather than selecting a laser source for a particular biological substrate as is current practice. The radiant energy source is then sufficient to destroy or carbonize stained tissues, which are attuned to absorb the energy from the source by the stain. The stain enhances absorption of incoming radiant energy, which results in increased and accelerated destruction of stained tissues. The increased absorption by stained tissues then reduces overpenetration into the column of tissues underlying the stained tissue. Therefore, this method provides clinicians with the ability to selectively mark a tissue for destruction, while leaving wanted tissues generally intact. The method also allows the most efficient laser to be used on any biological substrate regardless of the wavelengths produced. For example, a stain may be applied in a liquid form directly to selected biological tissues, followed by radiating the stained area with a laser that produces a wavelength that the stain readily absorbs. The method also incorporates the use of a radiant energy opaque substance that can be applied adjacent the stained treatment area to protect against accidental or incidental exposure to wanted tissue.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIGS. 1 through 17 are graphs of absorption spectra, depicting absorption intensity over light wavelength of sample stains, each figure and stain being listed below.

FIG. 1 is an absorption spectrum graph of amaranth.

FIG. 2 is an absorption spectrum graph of 8-anilinonaphthalene-1-sulfonic acid ammonium salt.

FIG. 3 is an absorption spectrum graph of bromophenol red (ph7).

FIG. 4 is an absorption spectrum graph of cresol red.

FIG. 5 is an absorption spectrum graph of 2, 7 dichlorofluroescein.

FIG. 6 is an absorption spectrum graph of eosin 4-isothiocyanate.

FIG. 7 is an absorption spectrum graph of eosin Y.

FIG. 8 is an absorption spectrum graph of FD&C Blue #1.

FIG. 9 is an absorption spectrum graph of FD&C Green #3.

FIG. 10 is an absorption spectrum graph of FD&C Yellow #5 (Tartrazine).

FIG. 11 is an absorption spectrum graph of methylene blue.

FIG. 12 is an absorption spectrum graph of naphthol blue black.

FIG. 13 is an absorption spectrum graph of nigrosin.

FIG. 14 is an absorption spectrum graph of neutral red.

FIG. 15 is an absorption spectrum graph of safranine O.

FIG. 16 is an absorption spectrum graph of thymol blue.

FIG. 17 is an absorption spectrum graph of xylenol blue.

### DETAILED DESCRIPTION OF THE PREFERRED EMBODIMENT

With reference now to the drawings, the preferred embodiment of the method is herein described. It should be noted that the articles “a”, “an” and “the”, as used in this specification, include plural referents unless the content clearly dictates otherwise.

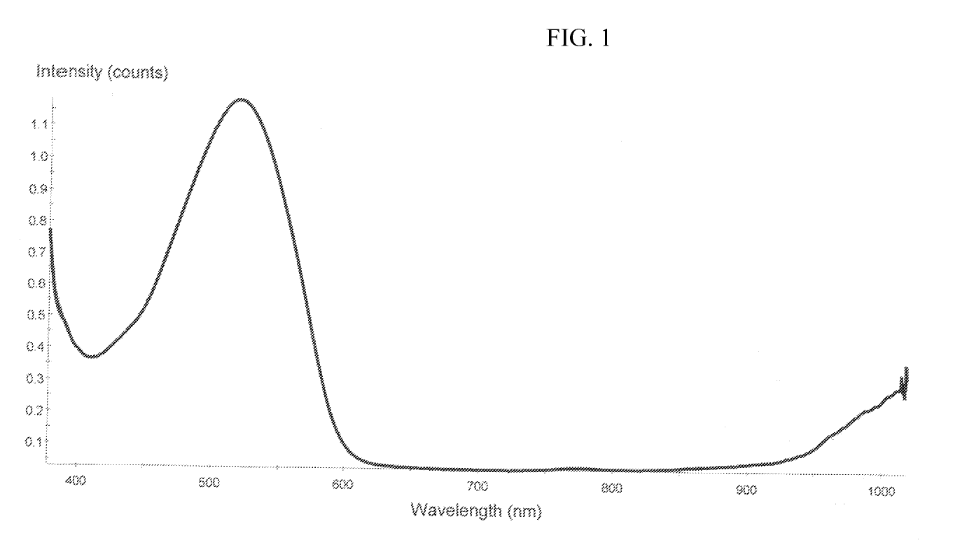

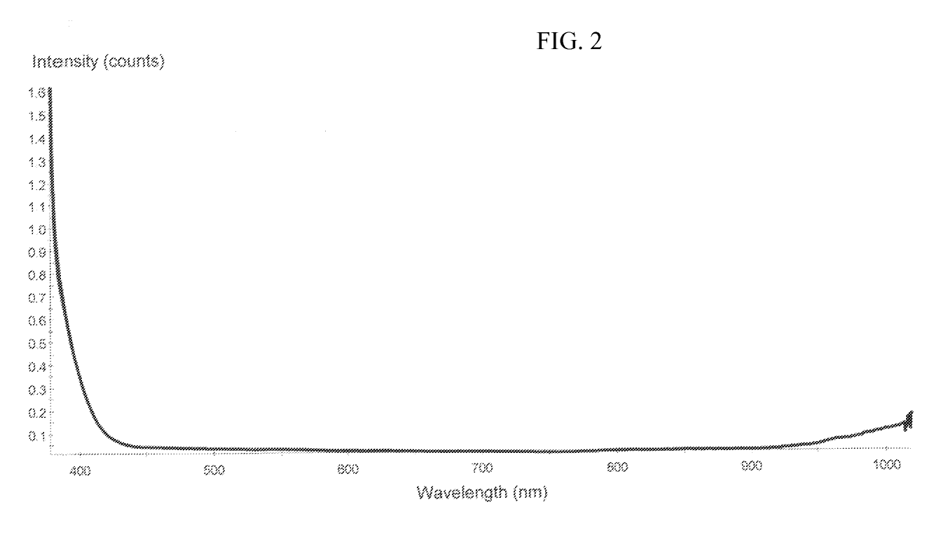

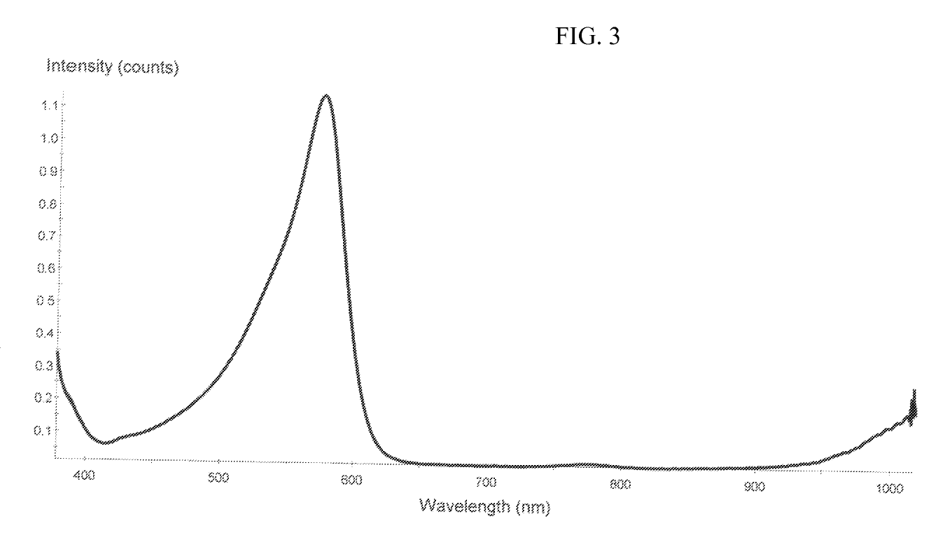

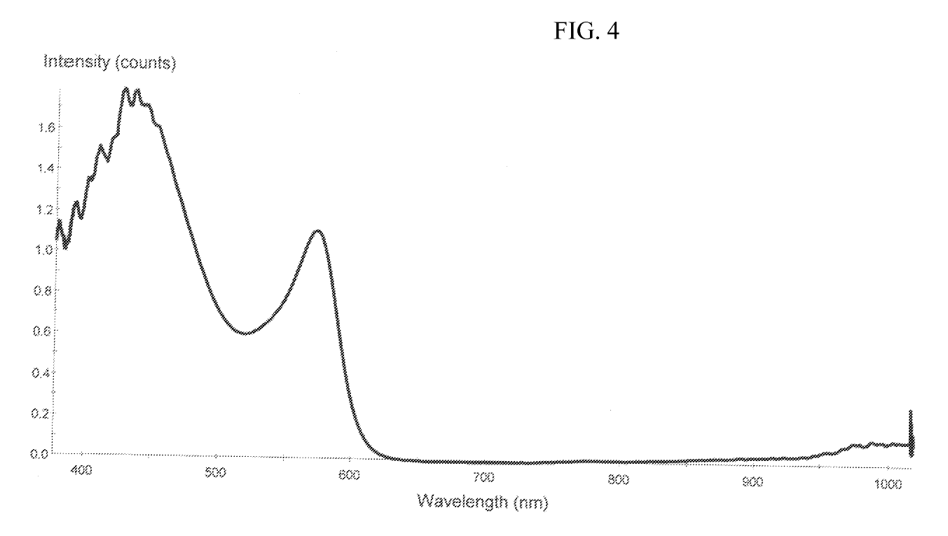

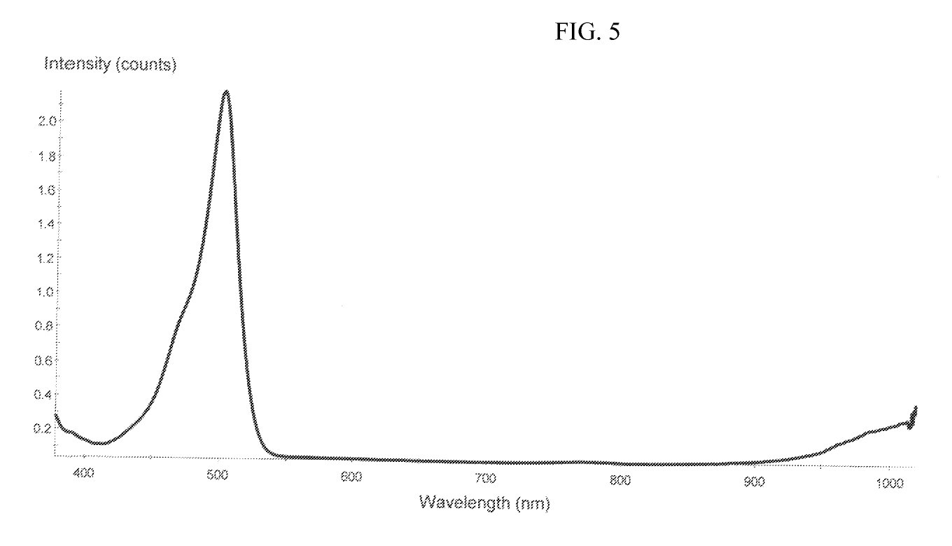

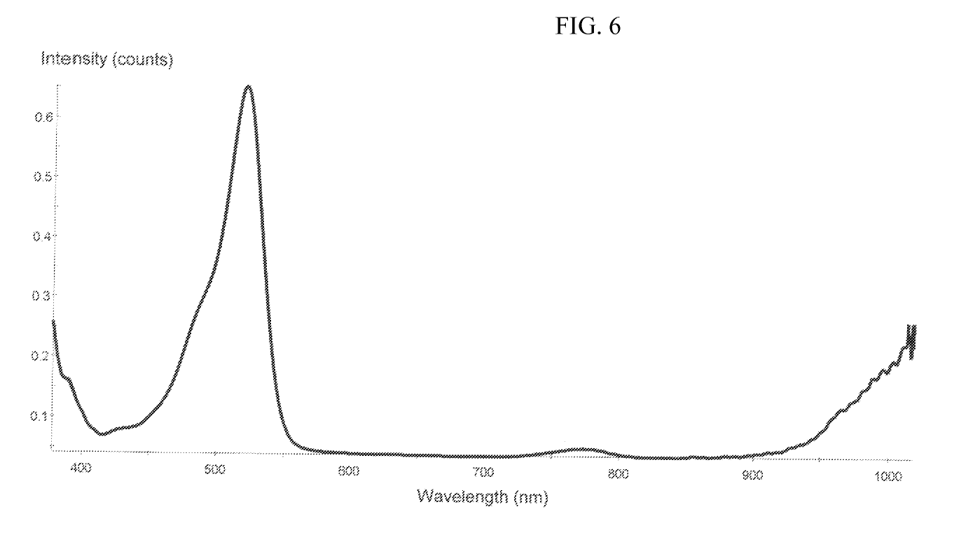

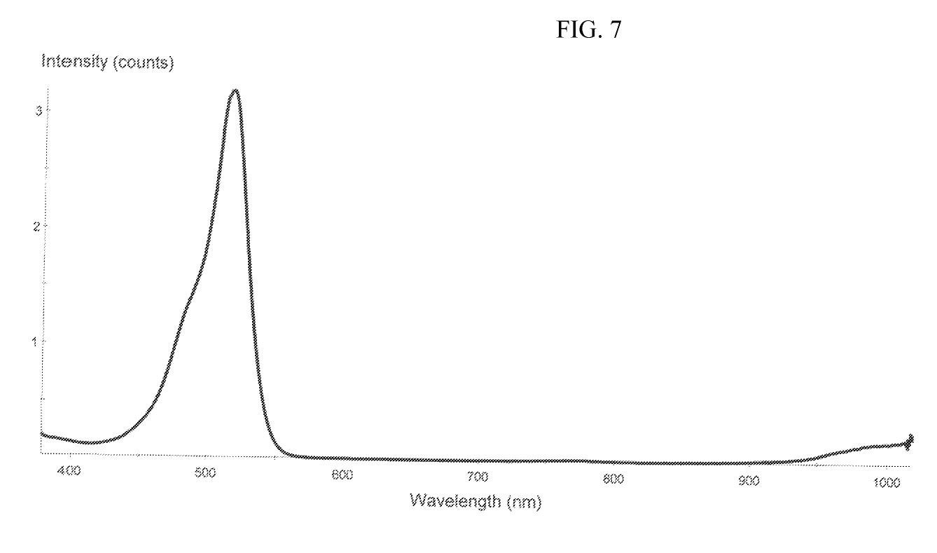

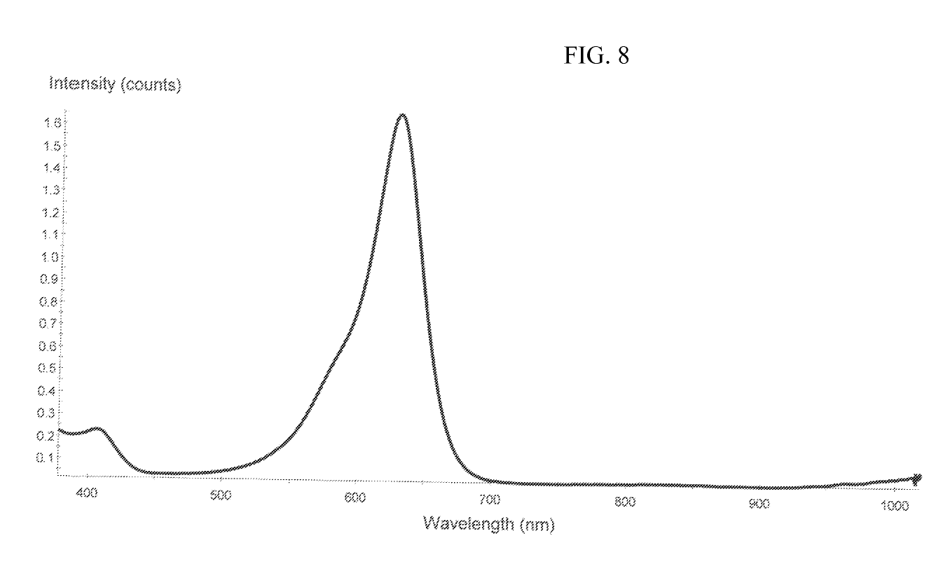

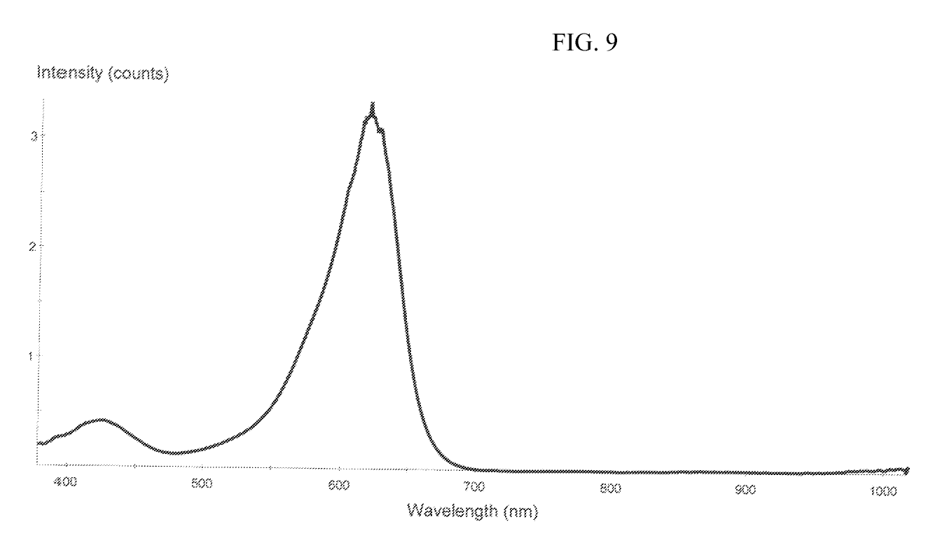

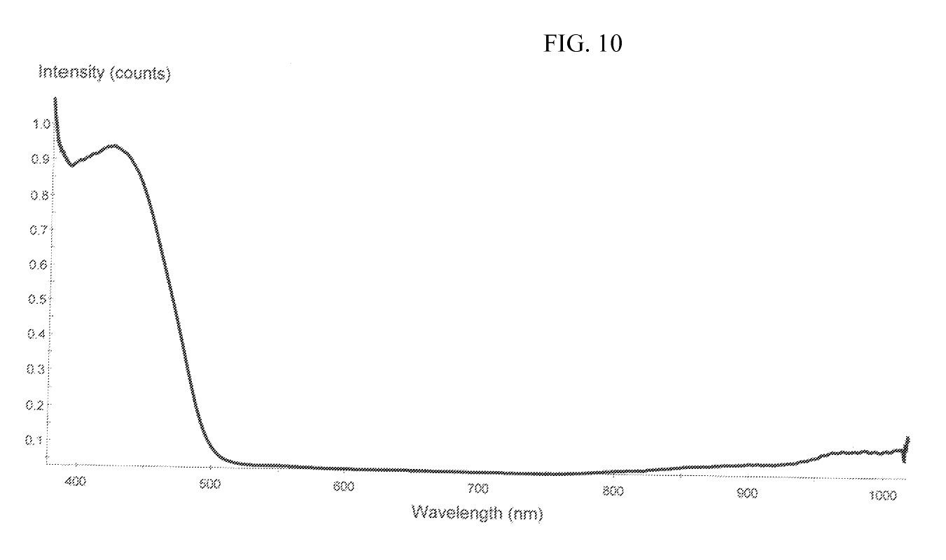

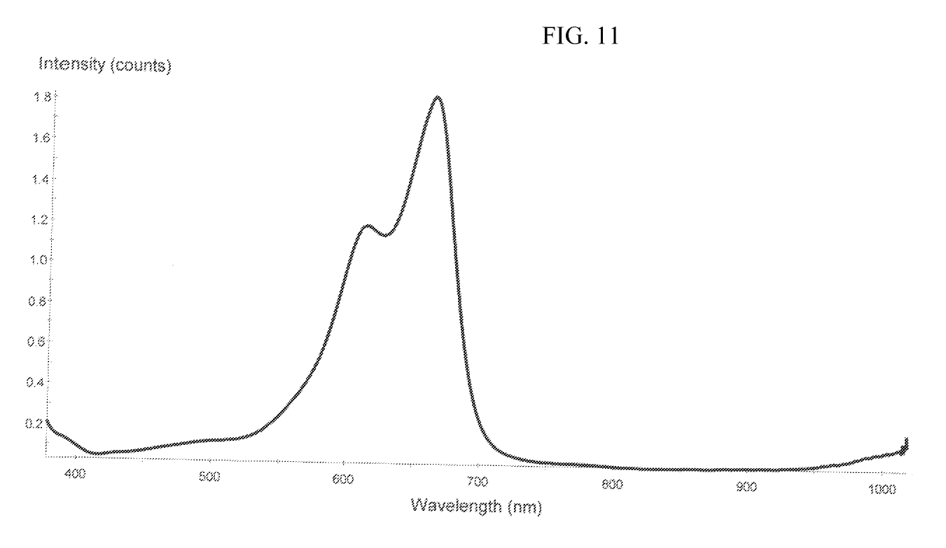

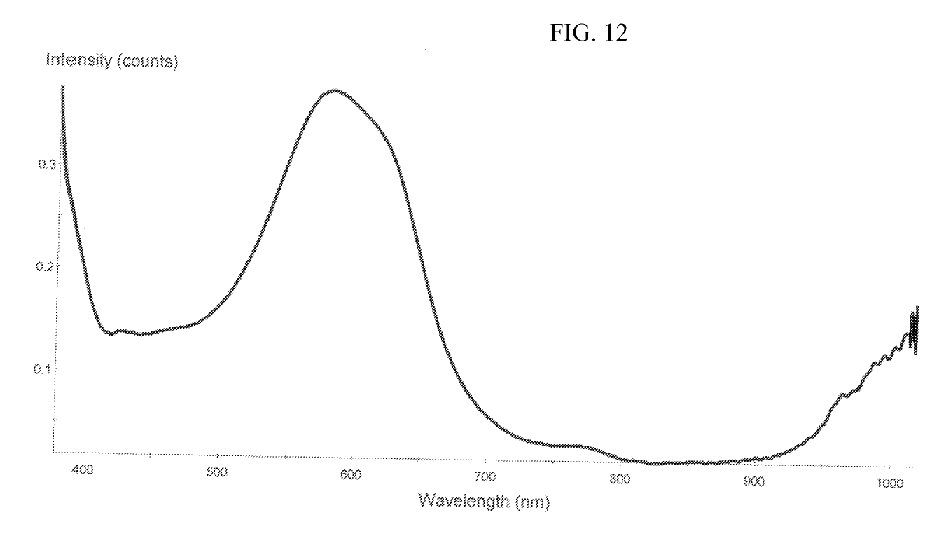

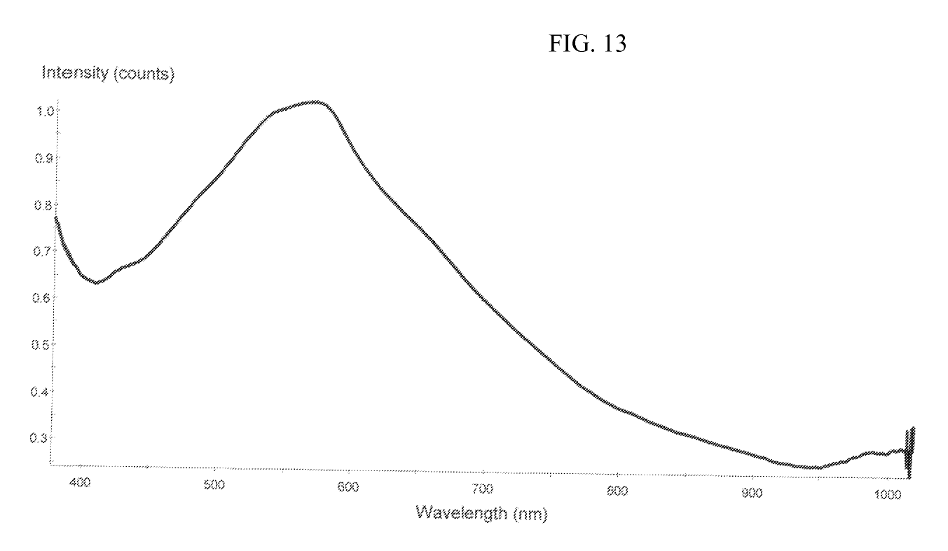

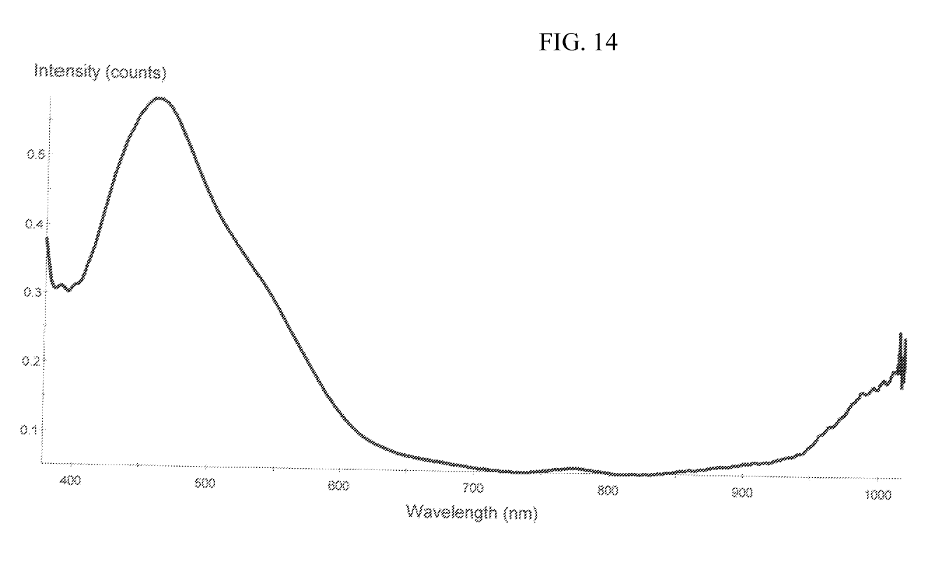

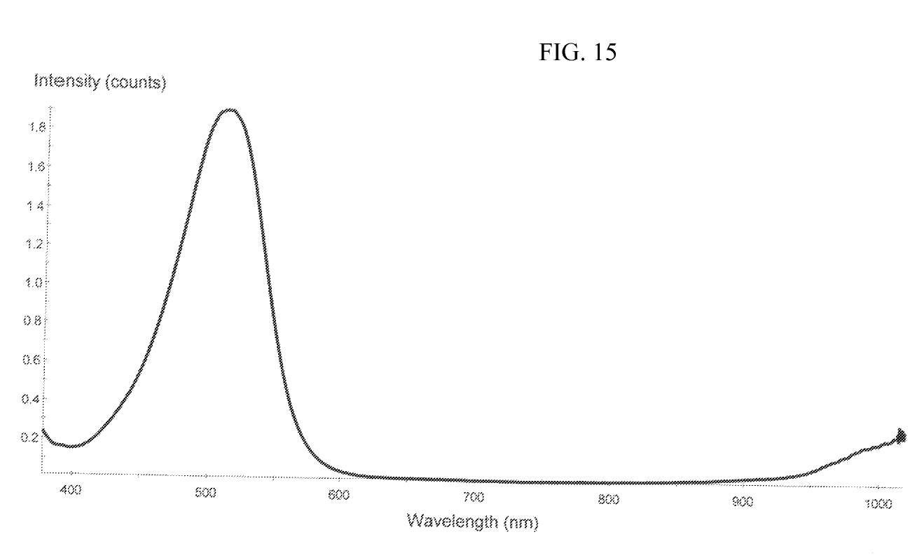

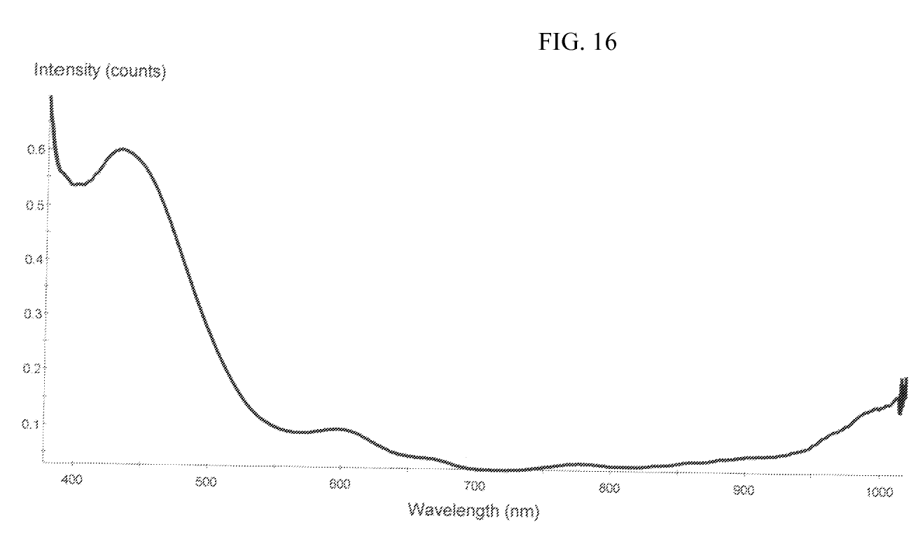

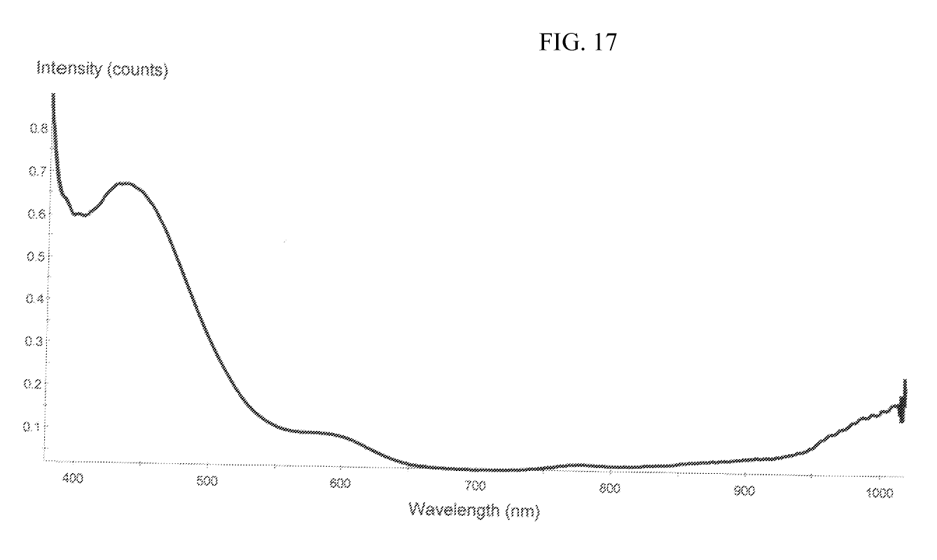

FIGS. 1-17 are examples of absorption spectra of various stains that could be used in the disclosed method. Comparing absorption spectra with the wavelength of a radiant energy source permits matching the source and stain for an efficient tissue cutting system and method. As shown in FIG. 1, the absorption spectrum for amaranth peaks at a wavelength of approximately 510 nm, the λ-max. Therefore, the use of a radiant energy source that has an energy output of 510 nm with the dye amaranth would be in accordance with the method herein disclosed. Likewise, FIGS. 2 through 17 are the spectra for sixteen other stains, each having at least one λ-max and each may be utilized with an energy source with an output having a wavelength corresponding to a given stain's λ-max. In a particular example of the practice of this method, it should be noted that diode lasers are capable of emitting energy with a wavelength of 810 nm. Indocyanine green, a particular stain that has been used extensively in other, unrelated, medical applications, has a λ-max of approximately 810 nm. The use of indocyanine green as an enhancing stain to aid in procedures where the practitioner uses a diode laser is firmly within the teachings of this method.

The method includes staining a selected tissue with a stain that is attuned to absorb the energy from a radiant energy source. The stain enhances absorption of incoming radiant energy, which results in increased destruction of stained tissues and the lessening of destruction of the column of tissues underneath the stained tissue. This method allows biological tissues to be destroyed by various strategies. Radiant energy can be concentrated to a degree as to totally annihilate a targeted biological tissue. Radiant energy can also be applied to damage tissues sufficiently that it will ultimately result in a scab, and be removed by natural events. The stain can be comprised of any substance with the ability to absorb or accept electromagnetic radiation from any radiant energy source. Radiant energy may be applied to an area from inside, arthroscopically, or outside the body.

In relation to tumors, which acquire their own blood supply independent from host tissue. Injection of a stain into the independent supply, stains the tumor alone and leaves the surrounding tissue unaltered. Treatment by radiant energy located anywhere in relation to the body may then be utilized. It should also be noted that some biological tissues are transparent to some forms of radiant energy, e.g. flesh vis-à-vis X-rays. In theory, it is possible to stain a biological tissue, even a tumor, so that it is no longer transparent to a particular form of energy and use that energy to treat the stained tissue without harming the surrounding tissue.

There are literally thousands of dyes, stains and pigments that are commercially available and could be used with the disclosed methods. A few examples of such dyes stains and pigments that may be used individually or as an ingredient in a staining compound include, but are not limited to, are: carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine O, FD&C carmine, rhodamine G, napthol blue black, D&C orange #4, thymol blue, auramine O, D&C red #22, D&C red #6, xylenol blue, chrysoidine Y, D&C red #4, sudan black B, D&C violet #2, D&C red #33, cresol red, fluorescein, fluorescein isothiocyanate, bromophenol red, D&C red #28, D&C red #17, amaranth, methyl salicylate, eosin Y, lucifer yellow, thymol, dibutyl phthalate, indocyanine green, and the like. The preferred stain is one that is generally deemed biologically compatible or non-toxic and may include any of the above dyes, pigments and stains as an ingredient in a final solution used as a stain. Other stains, currently existing or discovered or manufactured in the future, may be readily utilized in this method. Therefore, the above listing should not be considered definitive, but rather illustrative of stains to be utilized in the disclosed method and in no way be considered limiting.

One method of applying the stains to biological tissues to be cut or destroyed can be performed by placement of either a powdered or a liquid form directly on the tissues. This can be done by spreading or smearing a dried powder with a flat instrument over the biological tissue to be treated. The soluble stains can be dissolved in a solvent such as water, glycerin, propylene glycol, mineral oil, ethanol, acetone, polysorbate 80, or any like solvent. These dissolved stains can be applied to biological tissues by means of a brush, a syringe, a pen, a cotton pellet, or any fibrous material. Some stains may be a liquid without being dissolved by a solvent; these may also be applied by means of a brush, a cotton pellet, a syringe, a pen, or any fibrous material. Liquid stains may also be injected by means of a hypodermic needle and syringe or any other subcutaneous injection device to a target area beneath the surface of biological tissue. These internal treatment areas can be radiated orthoscopically or with any other subcutaneous method or device with radiant energy. These stains may optionally contain an anesthetic such as lidocaine, benzocaine, or any local or systemic anesthetic that would aid in alleviating any pain or discomfort caused by the procedure. If properly applied, the stain may additionally serve to map a practitioner's intended treatment area and plan, thereby serving a secondary purpose as well as enhancing the utility of the laser treatment. In current practice, the drawn features in a map serve no purpose other than to indicate where a practitioner is to cut and serve as a general guide to the treatment procedure.

These stains can be formulated into various compositions to best fit a medical, veterinary, or dental procedure, examples of which are presented below:

Example Formula #1

100%—nigrosin

Example Formula #2

1%—nigrosin

99%—water

Example Formula #3

100%—FD&C Blue #2

Example Formula #4

1.5%—FD&C Blue #2

98.5%—water

Example Formula #5

0.1%—FD&C Blue #2

30%—ethanol

69.9%—Water

Example Formula #6

1%—FD&C Green #3

30%—ethanol

69%—Water

Example Formula #7

2%—Cresol red

98%—ethanol

Example Formula #8

0.5%—amaranth

10%—ethanol

89.5%—glycerol

Example Formula #9

100% Amaranth

Example Formula #10

1%—Eosin 4-isothiocyanate

25%—Polyethylene glycol 600

74%—ethanol

Example Formula #11

99%—Bromophenol Red

1%—Water

Example Formula #12

1.0%—FD&C Yellow #5

99%—Glycerol

Example Formula #13

3%—FD&C Blue #2

10%—polysorbate 80

87%—Water

Example Formula #14

5%—Indocyanine Green

95%—Water

The above example formulas are all able to adequately stain biological tissue.

The methods for cutting or destroying tissue warrant use of a radiant energy source with sufficient energy to destroy, carbonize or pyrolize biological tissue. The radiant energy can be produced from sources such as high intensity light from incandescent, halogen or plasma arc devices. The radiant energy can be produced from sources such as solid-state lasers, examples of which are neodymium YAG, titanium sapphire, thulium YAG, ytterbium YAG, Ruby, holmium YAG lasers and the like. The radiant energy can be produced from sources such as EB or electron beam devices. The radiant energy can be produced from sources such as gas lasers, examples of which are the Carbon dioxide laser, argon gas, xenon gas, nitrogen gas, helium-neon gas, carbon monoxide gas, hydrogen fluoride gas lasers and the like. The radiant energy can be produced from sources such as a diode laser, examples of which are the gallium nitride, aluminum gallium arsenide diode laser and the like. There are also many dye lasers that utilize a radiant energy source that pass through various stains to achieve various wavelengths. Dye lasers are also within the scope of this method.

The method can include use of a radiant energy opaque substance that can be applied around the stained treatment area to protect against incidental or accidental exposure of harmful radiant energy during treatment. A typical procedure would begin by staining the area to be treated with a stain that is attuned to absorb the light from a radiant energy source, followed by covering the surrounding area with a substance that reflects or is opaque to the incoming radiant energy being produced. This combined procedure allows for targeted or selective destruction of biological tissues. The procedure allows the clinician to destroy or annihilate precisely the biological tissues selected and keep intact those tissues that are intended to remain.

A radiant energy opaque substance can be one that reflects most radiant energy and of a substance that is not combustible, for example, inorganic compounds that do not readily combine with atmospheric gases at elevated temperatures. Examples of radiant energy opaque substances are titanium dioxide, zinc oxide, calcium carbonate, and the like. Typically, radiant energy opaque substances are usually visibly white in color.

A method of applying the radiant energy opaque substance to biological tissues can be done by placement of the powdered form directly on the tissues. This can be done by spreading or smearing a dried powder with a flat instrument over the biological tissue to be treated. These substances can be blended in water to form a paste. These opaque suspensions can be applied to biological tissues by means of a brush, a flat instrument, a cotton pellet, a syringe, or any fibrous material. The paste can also contain a suspending aid to avoid settling of solids over time. Examples of suspending aids are sodium carboxy methylcellulose, fumed silica, sodium carboxy ethyl cellulose, precipitated silica, guar gum, and the like.

Radiant energy opaque substances can be formulated into various compositions to best fit a medical, veterinary, or dental procedure, an example of which is presented below:

Example Formula #1b

50%—powdered titanium dioxide

1%—sodium carboxy methyl cellulose

49%—water

The above example formula would be recognized as adequately able to cover and protect biological tissue from incidental harmful radiant energy.

Another variation of this method is to apply an oxidizing substance to the targeted area before use of the laser. An oxidizing substance is any substance that releases oxygen upon decomposition. The substance decomposes and releases oxygen into the immediately surrounding environment, thereby enhancing the destruction of the targeted tissue. The substance may be applied in addition to the stain or may be a component ingredient of the stain if maintained in a stable form. Oxidizing substances may be organic or inorganic. Potential oxidizing substances that may be utilized in this method include: benzoyl peroxide, T-butyl peroxide, T-butyl peroxide benzoate, potassium nitrate, potassium nitrite, potassium chlorate, potassium chlorite, sodium nitrate, sodium nitrite, sodium chlorate, and sodium chlorite. It should be noted, however, that the use of certain stains, such as indocyanine green, may be so efficient as to render the addition of an oxidizing substance superfluous.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method for targeted destruction of biological tissues by application of radiant energy, comprising:
a. A step of selecting a stain with an absorption spectrum corresponding to a wavelength of a given radiant energy source;
b. A step of applying the stain to a biological substrate to be destroyed; and
c. A step of utilizing the radiant energy source to energize the stained substrate until said substrate is destroyed;
Wherein the stained substrate acquires sensitivity to the wavelength of the radiant energy source from the stain.

2. The method of claim 1, the stain containing at least one ingredient selected from the group of ingredients consisting of: carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine O, FD&C carmine, rhodamine G, napthol blue black, D&C orange #4, thymol blue, auramine O, D&C red #22, D&C red #6, xylenol blue, chrysoidine Y, D&C red #4, sudan black B, D&C violet #2, D&C red #33, cresol red, fluorescein, fluorescein isothiocyanate, bromophenol red, D&C red #28, D&C red #17, amaranth, methyl salicylate, eosin Y, lucifer yellow, thymol, dibutyl phthalate, and indocyanine green.

3. The method of claim 1, the stain being applied to the selected biological substrate by hypodermic injection.

4. The method of claim 3, the stain further comprising an anesthetic.

5. The method of claim 1, the stain being applied to the selected biological substrate by spreading a paste containing the stain over the selected biological substrate.

6. The method of claim 5, the stain further comprising an anesthetic.

7. The method of claim 1, the stain being applied to the selected biological substrate by spreading a liquid containing the stain over the selected biological substrate.

8. The method of claim 7, the stain further comprising an anesthetic.

9. The method of claim 1, the stain being applied to the selected biological substrate by spreading a powder containing the stain over the selected biological substrate.

10. The method of claim 9, the stain further comprising an anesthetic.

11. The method of claim 1, the stain being applied to the selected biological substrate by utilizing a pen containing the stain to mark the substrate.

12. The method of claim 9, the stain further comprising an anesthetic.

13. The method of claim 1, further comprising the step of adding an oxidizing substance to the biological substrate.

14. The method of claim 13, the oxidizing substance being applied as a part of the stain.

15. The method of claim 13, the oxidizing substance being selected from the set of oxidizing substances consisting of: benzoyl peroxide, T-butyl peroxide, T-butyl peroxide benzoate, potassium nitrate, potassium nitrite, potassium chlorate, potassium chlorite, sodium nitrate, sodium nitrite, sodium chlorate, and sodium chlorite.

16. The method of claim 1, further comprising a step of applying a radiant opaque substance to tissues surrounding the biological substrate, wherein said surrounding tissues are then protected from absorbing energy from the radiant source.

17. The method of claim 16, the stain containing at least one ingredient selected from the group of ingredients consisting of: carbon black, FD&C Blue #2, nigrosin, FD&C black shade, FD&C blue #1, methylene blue, FD&C blue #2, malachite green, D&C green #8, D&C green #6, D&C green #5, ethyl violet, methyl violet, FD&C green #3, FD&C red #3, FD&C red #40, D&C yellow #8, D&C yellow #10, D&C yellow # 11, FD&C yellow #5, FD&C yellow #6, neutral red, safranine O, FD&C carmine, rhodamine G, napthol blue black, D&C orange #4, thymol blue, auramine O, D&C red #22, D&C red #6, xylenol blue, chrysoidine Y, D&C red #4, sudan black B, D&C violet #2, D&C red #33, cresol red, fluorescein, fluorescein isothiocyanate, bromophenol red, D&C red #28, D&C red #17, amaranth, methyl salicylate, eosin Y, lucifer yellow, thymol, dibutyl phthalate, and indocyanine green.

18. The method of claim 16, the stain being applied to the selected biological substrate by hypodermic injection.

19. The method of claim 18, the stain further comprising an anesthetic.

20. The method of claim 16, the stain being applied to the selected biological substrate by spreading a paste containing the stain over the selected biological substrate.

21. The method of claim 20, the stain further comprising an anesthetic.

22. The method of claim 16, the stain being applied to the selected biological substrate by spreading a liquid containing the stain over the selected biological substrate.

23. The method of claim 22, the stain further comprising an anesthetic.

24. The method of claim 16, the stain being applied to the selected biological substrate by spreading a powder containing the stain over the selected biological substrate.

25. The method of claim 24, the stain further comprising an anesthetic.

26. The method of claim 16, the stain further comprising an anesthetic.

27. The method of claim 26, the stain being applied to the selected biological substrate by utilizing a pen containing the stain to mark the substrate.

28. The method of claim 16, the radiant opaque substance containing at least one ingredient selected from the group of ingredients consisting of: titanium dioxide, zinc oxide, and calcium carbonate.

29. The method of claim 16, further comprising the step of adding an oxidizing substance to the biological substrate.

30. The method of claim 29, the oxidizing substance being applied as a part of the stain.

31. The method of claim 29, the oxidizing substance being selected from the set of oxidizing substances consisting of: benzoyl peroxide, T-butyl peroxide, T-butyl peroxide benzoate, potassium nitrate, potassium nitrite, potassium chlorate, potassium chlorite, sodium nitrate, sodium nitrite, sodium chlorate, and sodium chlorite.

32. A method for targeted destruction of biological tissues by application of radiant energy, comprising:
a. A step of electing a radiant energy source to which biological tissue is transparent;
b. A step of staining a targeted biological substrate so that it is not transparent to the radiant energy;
c. A step of utilizing the radiant energy source to energize the stained substrate until said substrate is destroyed;
Wherein the stained substrate acquires sensitivity to the wavelength of the radiant energy source from the stain.

33. The method of claim 32, further comprising the step of adding an oxidizing substance to the biological substrate.

34. The method of claim 33, the oxidizing substance being applied as a part of the stain.

35. The method of claim 33, the oxidizing substance being selected from the set of oxidizing substances consisting of: benzoyl peroxide, T-butyl peroxide, T-butyl peroxide benzoate, potassium nitrate, potassium nitrite, potassium chlorate, potassium chlorite, sodium nitrate, sodium nitrite, sodium chlorate, and sodium chlorite.
